(19) United States
(12) Patent Application Publication
(10) Pub. No.: US 2021/0016066 A1
(43) Pub. Date: Jan. 21, 2021

(54) IV CANNULA WITH TIP PROTECTOR ASSEMBLY

(71) Applicant: Lars Medicare Private Limited, Sonipat, Haryana (IN)

(72) Inventor: Paramjeet Singh, Sangam Vihar, New Delhi (IN)

(21) Appl. No.: 17/043,648

(22) PCT Filed: Mar. 29, 2019
(86) PCT No.: PCT/IN2019/050262

§ 371 Date: Sep. 29, 2020

(30) Foreign Application Priority Data
Mar. 31, 2018 (IN) 201811012276

(51) Int. Cl. A61M 25/06; A61M 25/00

(52) U.S. Cl. CPC A61M 25/0618; A61M 25/0097; A61M 25/0693; A61M 25/0631; A61M 25/0606

(57) ABSTRACT

The present invention relates to an IV cannula with tip protector assembly including a tip protector assembly slidably and coaxially arranged over a needle and having a locking mechanism which is automatically triggered during withdrawal of the needle after successful placement of the catheter tube in patient's vein. The tip protector assembly securely locks the tip and prevents it from coming out of either and of the tip protector. The tip protector assembly thereby prevents any possibility of needle stick injuries to the person placing the catheter tube inside the body of the patient, and hence preventing them from common needle stick hazards.

### CROSS REFERENCE TO RELATED APPLICATIONS

The present application is a § 371 national stage of International Application PCT/IN2019/050262, filed Mar. 29, 2019, which claims priority benefit of India Pat. Application Ser. No. 201811012276, filed Mar. 31, 2018, both of which are hereby incorporated herein by reference in their entireties.

### TECHNICAL FIELD

The present invention relates to intravenous cannula with tip protectors, and, more particularly to safety intravenous cannula having protection against accidental injury due to sticking of the sharp tip of the needle.

### BACKGROUND OF THE INVENTION

IV catheters are primarily used to infuse fluids, sometimes containing medications, directly into a patient's vascular system or to withdraw blood from a patient. A hand held placement device that includes a sharp tip needle is used to insert the catheter into a patient's vein. Such a device generally consists of a hollow-bore needle and an over-the-needle tubing of plastic, silicone, or like materials, used to access the lumen of a blood vessel of a patient.

The catheter is inserted at a shallow angle through the patient's skin into a peripheral blood vessel by a health worker using the hand held placement device. The needle is then withdrawn leaving the catheter in place either for direct hook up for transfusion of fluid or for later use.

Once proper placement is confirmed the health worker applies pressure to the blood vessel by pressing down on the patient's skin near the tip of the needle and catheter.

During this process of placing the IV catheter in the vascular system of the patient, the hollow-bore needle becomes blood contaminated, and when the blood vessel is accessed, the needle becomes blood filled.

In recent years, there has been great concern over inadvertent injuries caused by the “blood contaminated needles” to the medical professionals. This puts the medical professionals at great risk of being infected by the diseases which can be transmitted from infected person to the injured person through the contaminated needle. Thus, it is desirable to avoid contact of the patient's body fluid with the medical professionals by protecting the needle tip/bevel. This very danger to the safety of the medical personnel/health care worker and others has caused an impetus for the invention of a safer intravenous Cannula in which the occurrence of such inadvertent injury due to needle stick will be prevented.

Various attempts have been made to prevent needle stick injuries through various kinds of assemblies of devices. However, some of them are very expensive and complex due to their large numbers of components, and some, despite solving the problems, take up a substantial amount of space. The prior art safety devices all exhibit one or more drawbacks that have thereby far limited their usefulness and full acceptance by health care workers.

The invention disclosed in U.S. Pat. No. 4,026,287 provides for retraction of a used needle into a cavity in a unitary, sturdy structure. However, it requires screwing the syringe plunger into the back of the needle flange after use, to destroy a fragile seal around the flange, and then retract the needle. There are also chances of inadvertent introduction of fingertips into the syringe barrel. Even the plunger could remain in place, held only by detents at the rear of the barrel.

In U.S. Pat. No. 4,935,012, a safety device comprises an elongated protective sleeve supported by front and rear bearing members for sliding movement about a medical needle and its associated support structure such as a support hub or barrel. The protective sleeve is slidable in a stable manner from a first position with the pointed end of the needle safely retracted within and shielded by the front bearing member to a second position with the needle projecting through and beyond the front bearing member for normal use. After use, the sleeve is slidable to a third position derailing the pointed needle end from the front bearing member, and exposing a visual indicator to indicate that the needle has been used. Closure flaps associated with the front bearing member, and thereby prevent accidental or unauthorized needle reuse. Dent members interacting between the rear bearing member and the needle support structure releasably lock the protective sleeve in the various positions.

The invention of U.S. Pat. No. 4,935,012 has its own limitations. The said invention provides a slidable sleeve for the protection of the needle which requires mechanical movement, such that skipping of mechanically moving the protection sleeve over the needle after the use may lead to needle stick injury. Further, the slidable sleeve is not provided with any mechanism to prevent the needle from coming out of it if both are pulled in opposite directions.

### SUMMARY OF THE INVENTION

Certain terminology is used herein for the convenience only and is not to be taken as a limitation to the present invention. The term “distal” and “proximal” refer respectively to directions farther away and closer to the person administering the IV Cannula into the body of a patient. The terminology includes the words specifically mentioned, derivatives thereof and words of similar import.

The present invention provides for automatic protection against needle stick injuries, including a resilient material that prevents the forward movement of a needle if a push force is applied from behind the needle. According to aspect of the present invention a tip protector assembly is used within the IV Cannula assembly, which is economical because of its simple yet highly workable structure and it successfully protects the tip of the completely retracted needles ready for disposal.

The present invention envisages an intravenous cannula with safety tip protector assembly including a needle, an intravenous delivery catheter assembly removably located coaxially over the needle such that the needle can be withdrawn from the assembly after successful placement of the delivery end of the catheter tube in a patient's vein. The present invention includes a safety tip protector assembly, which is coaxially and slidably arranged over the needle, and removably attached to a proximal portion of the catheter assembly and adopted to protectively cover the needle tip and having mechanism for obstructing the needle tip from emerging out from a distal end of the safety tip protector assembly once retracted and activating the mechanism. The safety tip protector assembly also has mechanisms to engage a bulge present at the distal portion of the needle near the bevel, thereby preventing the portion of the needle from the bulge to the tip from emerging out from the proximal end of the safety tip protector assembly.

The safety tip protector assembly may be made up of non reactive metal, preferably stainless steel, and may be made from a retaining strip acting as a base and is present with a needle through bore, a stem which is an extension of one side of the base in a distal direction and further bending in a direction parallel to the base and having a needle through bore upon the parallel portion in such a manner that the needle through bores provide a passage for the needle. The needle through bore of the retaining strip has a profile larger than a principle profile of the needle but lesser than an outer profile of the bulge present on the needle. The needle through bore present at the parallel portion has a profile slightly larger than the outer profile of the bulge and allows the bulge to pass through it. The stem has a length lesser than the length of the needle portion between the bulge and the tip.

Optionally, the safety tip protector assembly has a first arm and a second arm which are each extensions of the sides of the base adjacent to the side attached with the stem in a distal direction. A plurality of claws are located at the distal ends of the first arm and the second arm respectively and adapted to prevent the tip of the needle from emerging out of the distal end of the tip protector assembly once the tip of the needle is retracted beyond the claws.

The safety tip protector assembly may also be provided with means adapted to engage a corresponding locking means present on the inside surface of the catheter hub assembly for removably holding the tip protector assembly within the catheter hub assembly.

The intravenous cannula with safety shield assembly may further include a protector slidably arranged over the catheter tube, wherein the proximal portion of the protector is removably attached to an attachment site at a distal portion of a wing body through a locking mechanism and hence protectively covering the tip as well as the catheter tube.

These and other objects, advantages and features of the invention will become apparent upon review of the following specification in conjunction with the drawings.

### BRIEF DESCRIPTION OF DRAWINGS

The invention is illustrated by way of example and not by way of limitation in the figures of the accompanying drawings in which like references indicate similar elements.

FIG. 1 is a top view of a complete assembly of a preferred embodiment according to the present invention;

FIG. 2 is a perspective view of the complete assembly of the preferred embodiment according to the present invention;

FIG. 3 is a cross sectional view of the complete assembly of the preferred embodiment according to the present invention;

FIG. 4 is an exploded view of the complete assembly of the preferred embodiment according to the present invention;

FIG. 5 is a cross sectional view of the catheter assembly;

FIG. 6 is a diagram of the needle;

FIG. 7 is a diagram of the safety tip protector assembly;

FIG. 8 is a diagrammatic representation of the needle in retracted position, before the tip of the needle passes the claws of the tip protector assembly;

FIG. 9 is a diagrammatic representation of the needle in retracted position after the tip passed the claws of the tip protector assembly, but before the tip protector assembly securing the tip is unlocked with the catheter assembly;

FIG. 10 is a diagrammatic representation of the tip protector assembly securing the tip of the needle and unlocked with the catheter assembly;

FIG. 11 is a diagrammatic representation of retracted needle hub assembly along with the tip protector assembly securely covering the tip of the needle; and

FIG. 12 is an isometric diagrammatic representation of retracted needle hub assembly along with the tip protector assembly securely covering the tip of the needle.

### DETAILED DESCRIPTION OF THE PREFERRED EMBODIMENTS

This description is not intended to be a detailed log of all the possible ways in which the invention may be implemented, or all features that may be added to the instant invention. For example, features illustrated with respect to a particular embodiment may be incorporated into another embodiment, and features illustrated with respect to a particular embodiment may be deleted from that embodiment. In addition, numerous variations and additions to the various embodiment suggested herein will be apparent to those skilled in the art in light of the present disclosure, which do not depart from the present invention. Hence, the following specifications are intended to illustrate some particular embodiment and variations thereof.

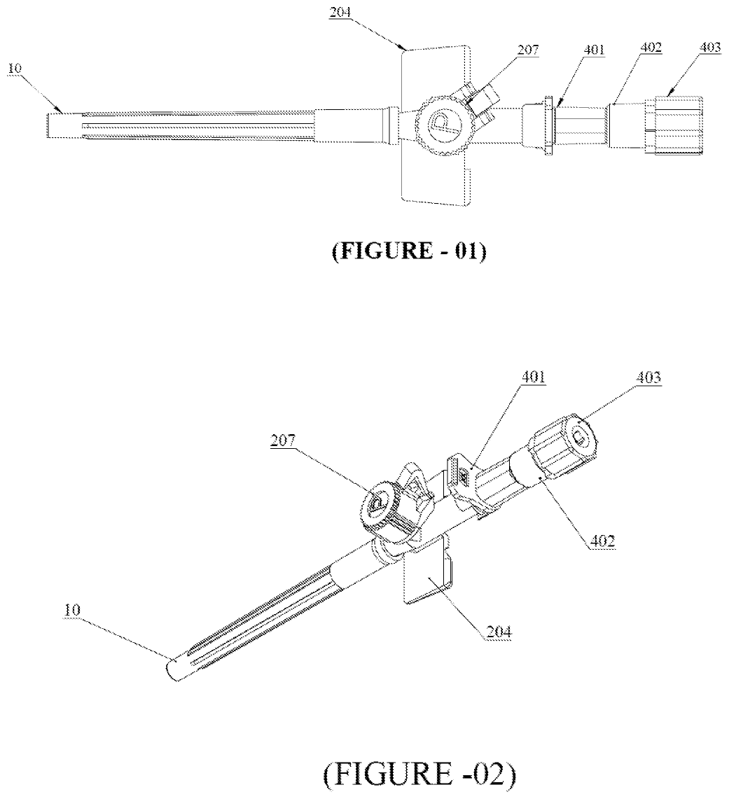

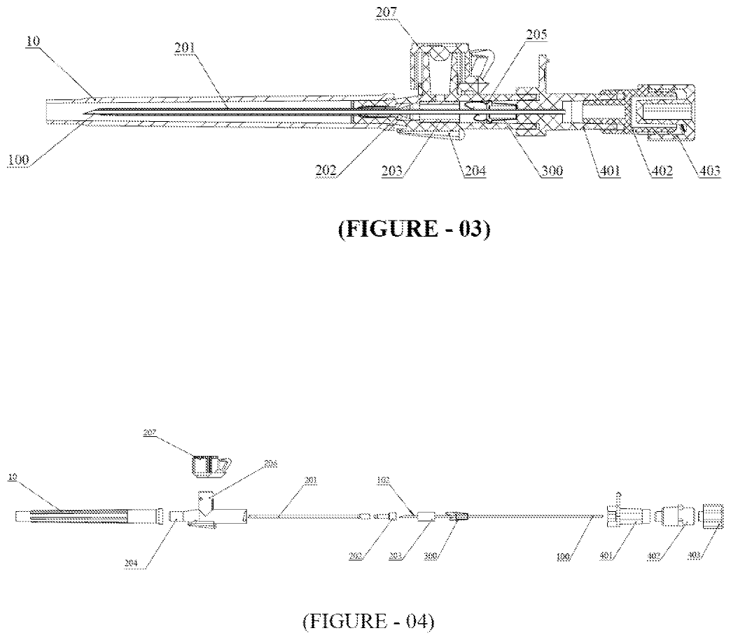

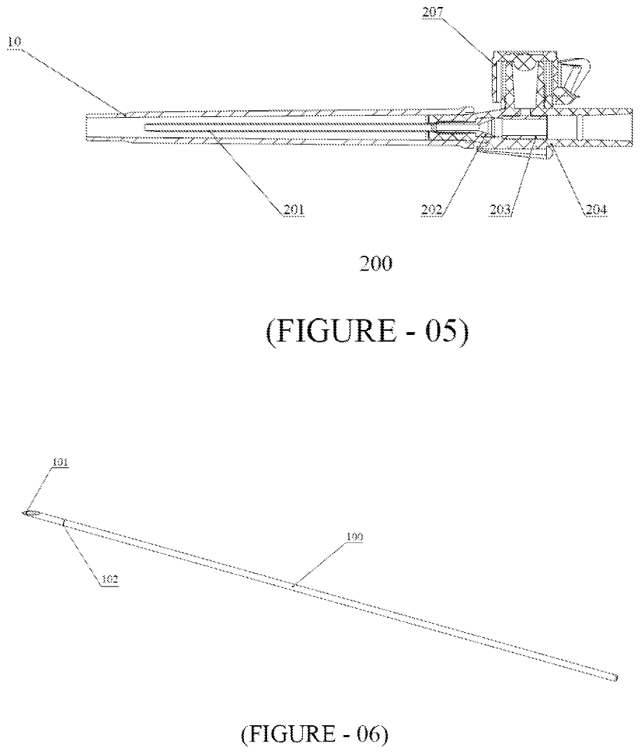

In a preferred embodiment of the present invention as referred in FIGS. 1 to 5, an intravenous delivery catheter assembly 200 is removably located coaxially over a needle 100 such that the needle 100 can be withdrawn from the assembly 200 after successful placement of the delivery end of the catheter tube 201 in a patient's vein. The intravenous delivery catheter assembly 200 further comprises a catheter tube 201, a catheter holder 202, a silicone tube 203, a wing body 204, wherein a safety tip protector assembly 300 coaxially arranged over the needle 100 is removably arranged inside the wing body 204 and is removably held with an inter locking mechanism attained with the engagement of a means, e.g. protruding rib pattern 205, present circumferentially upon the inside surface of the wing body 204 with corresponding means present upon the body of the tip protector assembly 300 adapted to lock itself with the rib pattern 205. The interlocking mechanism may be disengaged with the application of force in the proximal direction. A cover 10 is slidably arranged over the catheter tube 201 and is removably attached with the catheter hub/wing body 204 thereby protectively covering the bevel/tip 101 as well as the catheter tube 201. The assembly further includes an optional port 206 along with a port cap 207. A needle hub assembly 400 is coaxially mounted over the proximal end of the needle 100 and consist essentially of a needle hub 401, a flash back chamber 402 attached at the proximal portion of the needle hub 401 and a luer lock 403 releasably attached at the distal portion of the flash back chamber 402. The distal end of the needle hub assembly 400 is present with an elongation 404 adapted to push the safety tip protector assembly 300 in distal direction from its proximal end.

As shown in FIG. 6, the needle 100 also has a bulge 102 in the distal section of the needle and more specifically near the tip 101. The bulge 102 has an outer profile, the dimension of which is larger than maximum dimension of the principal outer profile of the needle 100. Optionally, a dent may be formed or established in the needle 100 at the location of the bulge 102.

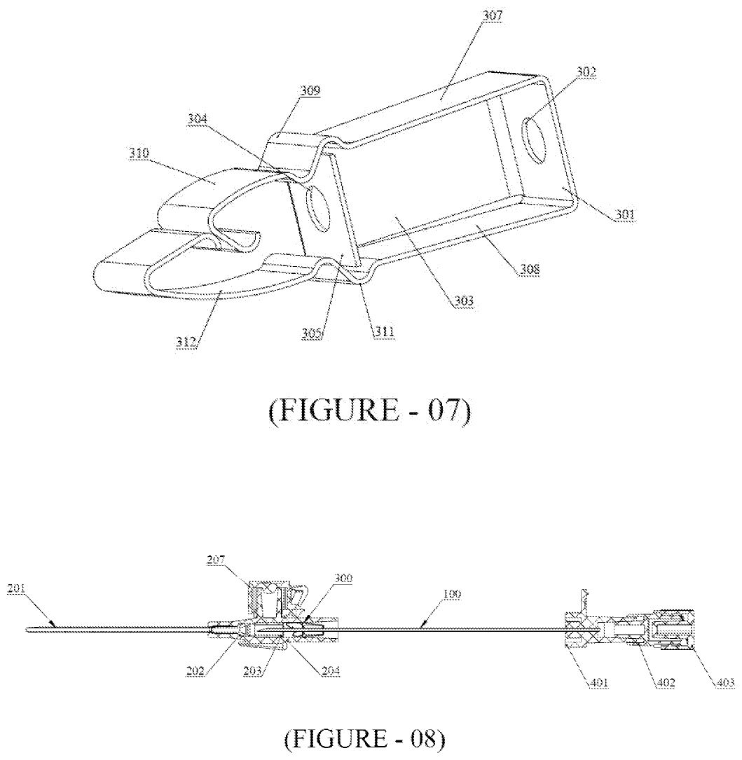

As shown in FIG. 7, the body of the tip protector assembly 300 of the present invention is made of non reactive metal, preferably but not limited to stainless steel. The tip protector assembly 300 includes a retaining strip, preferably a rectangular/square shape, acting as a base 301 and defining a proximal needle through bore 302. A stem 303, which is an extension of one side of the base 301 in a distal direction and further bending in a direction parallel to the base portion, is present with a distal needle through bore 304 upon the parallel portion 305 in such a manner that the needle through bores 302, 304 provide a passage 306 for the needle 100. The proximal needle through bore 302 has a profile larger than the principle profile of the needle 100 but lesser than the outer profile of the bulge 102 present on the needle 100. The distal needle through bore 304 has a profile slightly larger than the outer profile of the bulge 102 and allows the bulge 102 to pass through it. The stem 303 has a length lesser than the length of the needle portion between the bulge 102 and the tip 101. The tip protector assembly 300 further includes a first arm 307 and a second arm 308, which are extensions of the sides of the base 301 adjacent to the side attached with the stem 303 in a distal direction.

The first arm 307 of the tip protector assembly 300 has an engaging portion or means 309 adapted to engage the corresponding locking means, e.g. rib pattern 205, that is present on the inside surface of the catheter hub assembly 200 for removably holding the tip protector assembly 300 within the catheter hub assembly 200 in such a manner that it can be easily removed merely by applying pressure outwardly thereto. The first arm 307 further bends at an angle after the engaging means 309 and further extended in the angular direction in such a manner that the claw 310 present at the end of the first arm blocks the pathway of the needle 100 along the axial direction to the needle passage 306 and distal to the distal needle through bore 304. The claw 310 is an extension of the first arm 307 in which the end part of the first arm is bent twice in such a manner that the first arm forms a claw like structure so that once the tip 101 is inside the claw-like structure, the tip is effectively trapped inside and cannot escape even after being pushed further in the distal direction. It is pertinent to mention that the first arm 307 is sufficiently long enough so as to block the needle pathway beyond the tip 101 of the needle 100 while the bulge 102 is engaged with the proximal needle through bore 302; hence the path of the tip 101 of the needle 100 is blocked by the claw 310 when the needle 100 is pushed in the distal direction. The second arm 308, similar to the first arm 307, also extends distally in an axial direction and also has an engaging portion or means 311 adapted to engage with the rib 205 present inside the catheter hub assembly 200. The position of the engaging means 311 on the second arm 308 is opposite to the position of the engaging means 309 on the first arm 307 so that both arms may be simultaneously engaged with the rib 205 so as to provide efficient locking. The second arm 308 has another bend after the engaging means 311 at a certain angle towards the axis longitudinal to the needle passage 305. The second arm 308 has a sufficient length such that the claw 312 present on the second arm 307 blocks the needle path above the claw 310 of the first arm 307.

The tip protector assembly 300 is arranged such that the movement of the needle 100 in the proximal direction beyond the bulge 102 portion is stopped by the proximal needle through bore 302 present at the base portion 301 while a small portion of the needle, preferably a portion distal to bevel/tip, 101 still remains beyond the distal side of the distal needle through bore 304 present on the extended parallel portion 305 of the stem 303, ensuring movement of the needle 100 along one trajectory only passing through both needle through bores 302, 304.

While arranging the tip protector assembly 300 along the needle 100, the proximal end of the needle 100, slightly parting both arms 306, 307 of the tip protector assembly 300, is inserted from the distal needle through bore 304 present on the parallel portion 305 and further travels to the proximal needle through bore 302 present on the base 301.

The needle hub assembly 400 is then affixed with the proximal end of the needle 100. The slightly parted arms 307, 308 are thus stored with spring action restoring force. The needle 100 along with the tip protector assembly 300 is then arranged with the catheter assembly 200 by inserting the tip 101 of the needle from the rear portion of the catheter hub assembly 200 and the needle 100 is pushed further in the distal direction so that the needle tip 101 travels all along the catheter assembly and finally comes out at the end of the catheter tube 201. The elongation 404 present at the distal end of the needle hub assembly 400 is long enough to push the tip protector assembly 300 to such an extent inside the catheter hub assembly 200 so that the engaging portions or means 309, 311 present on the arms 307, 308 become removably attached with the ribs (205 present in the catheter hub 200. Any further movement of the projection 404 is stopped due to engagement of the proximal end of the wing body 204 with the distal end of the needle hub assembly upon which the elongation 404 is mounted. The means 309,311 present on the arms 306,307 of the tip protector assembly 300 become engaged with the ribs 205 present in the catheter hub assembly 200, thereby removably locking the shield assembly within the catheter hub assembly 200 in a releasable manner.

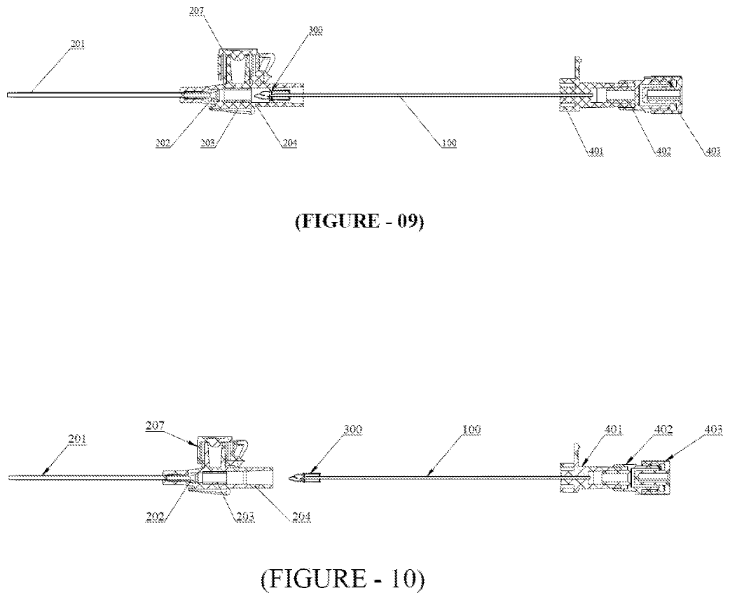

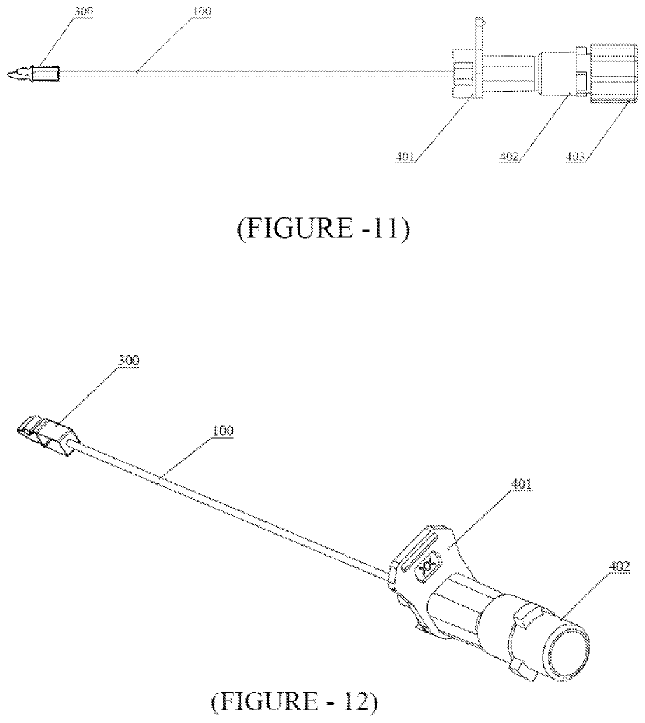

Referring to FIGS. 8-12, to use the IV Cannula with the tip protector assembly 300 on a patient, the needle cover 10 is first removed while holding the cannula by the wing body 204 so that the needle 100 along with the tip protector assembly 300 does not get pulled out from the catheter hub 204. After removing the needle cover 10, the IV cannula is used over the patient by a medical practitioner to puncture the vein of the patient. After successful puncture of the vein by the tip 101 of the needle 101 and confirmed by the flow of blood in the flash chamber 402, the needle hub assembly 400 is slowly pulled out with one hand while holding the catheter hub assembly 200 securely in place with the second hand. The needle then slowly travels backwards leaving catheter tube and the tip 101 then passes firstly through the wing body/catheter hub and then passes the claw 312 of the second arm 308 and then the claw 310 of the first arm 307. The needle 100 then travels further in the proximal direction until the bulge 102 becomes engaged with the proximal needle through bore 302. The moment the tip 101 passes the claws 312,310 of the arms 308,307 the restoring force present in the arms 308,307 makes them move in the transverse direction towards the needle pathway. The claws 312,310 present on arms 308,307 then become positioned so as to block the movement of the needle 100 in the distal direction. Further pulling force applied on the needle hub assembly 400 in the proximal direction pulls the tip protector assembly 300 out of the catheter hub assembly 200 whereby the tip 101 of the needle gets secured by the tip protector assembly 300 without any risk or danger of needle prick to the medical practitioner. The used needle inside the locked tip protector assembly 300 can then be safely disposed of.

Changes and modifications in the specifically-described embodiments may be carried out without departing from the principles of the present invention, which is intended to be limited only by the scope of the appended claims as interpreted according to the principles of patent law including the doctrine of equivalents.

1. An intravenous cannula comprising:
a needle having a distal end portion with a bevel/tip and a bulge, the bulge located nearer a proximal end;
an intravenous delivery catheter assembly removably located coaxially over the needle in such a manner that the needle can be withdrawn from the catheter assembly after successful placement of a delivery end of a catheter tube in a patient vein;
a tip protector assembly coaxially and moveably arranged over the needle and removably attached to a proximal portion of the catheter assembly and adapted to protectively cover the bevel/tip;
the tip protector assembly having a mechanism for obstructing the bevel/tip from emerging out of a distal end of the tip protector assembly once the bevel/tip is retracted and the mechanism is activated, and the tip protector assembly also having another mechanism to engage the bulge and prevent the portion of the needle from the bulge to the bevel/tip from emerging out of a proximal end of the tip protector assembly.

2. The intravenous cannula of claim 1, wherein the tip protector assembly comprises a non reactive metallic body.

3. The intravenous cannula of claim 1, wherein the tip protector assembly further comprises:
a retaining strip forming a base defining a proximal through-bore;
a stem extending from one side of the base in a distal direction and having a parallel portion bent in a direction parallel to the base and defining a distal through bore upon the parallel portion in such a manner that the proximal and distal through bores provide passage for the needle;
the proximal through bore having a profile larger than a principle profile of the needle but lesser than an outer profile of the bulge;
the distal through-bore having a profile larger than the outer profile of the bulge, allowing the bulge to pass through the distal through bore;
the stem having a length lesser than the length of the needle portion between the bulge and the bevel/tip;
a first arm and a second arm each extending distally from respective other sides of the base that are adjacent to the one side attached with the stem; and
a respective claw at a distal end of each of the first arm and the second arm, the claws adapted to prevent the bevel/tip of the needle from emerging out of the distal end of the tip protector assembly once the bevel/tip is retracted beyond the claws.

4. The intravenous cannula of claim 3, wherein the first and second arms of the tip protector assembly further comprise respective first and second engaging portions adapted to engage a corresponding rib present inside the catheter assembly to provide secure but removable holding of the tip protector assembly within the catheter assembly.

5. The intravenous cannula of claim 1, wherein the catheter assembly further comprises a catheter tube, a catheter holder, a silicone tube, and a wing body that is coaxially and removably attached over the tip protector assembly by a locking mechanism.

6. The intravenous cannula of claim 1, further comprising a needle hub assembly coaxially mounted over the needle and comprising a needle hub, a flash back chamber attached at a proximal portion of the needle hub, and a luer lock releasably attached at a distal portion of the flash back chamber, wherein a distal portion of the needle hub assembly has an elongation adapted to push the tip protector assembly inside the catheter assembly to the extent that first and second engaging portions of the tip protector assembly become removably interlocked with a corresponding rib present inside the catheter assembly.

7. The intravenous cannula of claim 5, further comprising a cover slidably arranged over the catheter tube, wherein a proximal portion of the cover is removably attached to an attachment site at a distal portion of the wing body through a locking mechanism to protectively cover the bevel/tip and the catheter tube.

8. The intravenous cannula of claim 4, wherein the catheter assembly further comprises a catheter tube, a catheter holder, a silicone tube, and a wing body that is coaxially and removably attached over the tip protector assembly by a locking mechanism.

9. The intravenous cannula of claim 8, further comprising a needle hub assembly coaxially mounted over the needle and comprising a needle hub, a flash back chamber attached at a proximal portion of the needle hub, and a luer lock releasably attached at a distal portion of the flash back chamber, wherein a distal portion of the needle hub assembly has an elongation adapted to push the tip protector assembly inside the catheter assembly to the extent that first and second engaging portions of the tip protector assembly become removably interlocked with a corresponding rib present inside the catheter assembly.

10. The intravenous cannula of claim 9, further comprising a cover slidably arranged over the catheter tube, wherein a proximal portion of the cover is removably attached to an attachment site at a distal portion of the wing body through a locking mechanism to protectively cover the bevel/tip and the catheter tube.
